(19) United States
(12) Patent Application Publication
(10) Pub. No.: US 2019/0125267 A1
(43) Pub. Date: May 2, 2019

(54) PHYSIOLOGICAL SIGNAL MONITORING APPARATUS

(71) Applicant: K-JUMP HEALTH CO., LTD., New Taipei City (TW)

(72) Inventor: CHAO-MAN TSENG, New Taipei City (TW)

(21) Appl. No.: 15/802,126

(22) Filed: Nov. 2, 2017

(51) Int. Cl. A61B 5/00; A61B 5/01

(52) U.S. Cl. CPC A61B 5/6843; A61B 5/01; A61B 5/681; A61B 5/721; A61B 2562/0271; A61B 5/0008; A61B 2560/0252; A61B 2560/0443; A61B 5/746

(57) ABSTRACT

A physiological signal monitoring apparatus includes at least one connection assembly and a physiological signal monitoring device. The connection assembly includes a first connecting body and a second connecting body. The physiological signal monitoring device is detachably combined with a fixing portion through the connection assembly. When the fixing portion is mounted on a living body, the physiological signal monitoring device at least monitors a temperature change and a displacement change of the living body. The physiological signal monitoring device includes an engaging member and a contact member. The engaging member at least partially matches with the second connecting body. The contact member elastically protrudes from an opening of the engaging member and is used for temperature sensing. The second connecting body is for being fixed at an outer side of the connecting region of the fixing portion so as to combine with the first connecting body.

### FIELD OF THE INVENTION

The disclosure relates in general to a physiological signal monitoring device, and more particular to a physiological signal monitoring apparatus detachably mounted to a living body.

### BACKGROUND OF THE INVENTION

For a conventional physiological signal monitoring device, a sensor needs to be tightly adhered to a user. When the number of categories of physiological signals that the physiological signal monitoring device needs to detect increases, an area of the sensor tightly adhered to a user also increases. Further, in an application scenario where precision is required, e.g., detection of a body surface temperature of a human body, the number of sensors and the covered parts also need to be increased. However, such configuration of sensors inevitably results in user discomfort. Further, if detection of physiological signals is needed for an extended period of time, the above method can cause user inconvenience and discomfort. During a period of rest or sleep of a user, the user may also unconsciously dislocate the sensors, thus failing the goal of detection.

Further, in certain application scenarios, e.g., detection of physiological signals of babies, children and elder persons, these users may become emotional or unwilling if a physiological signal monitoring device provides poor comfort. Therefore, there is a need for a solution that minimizes a contact area between a sensor of a physiological signal monitoring device and a user body while providing enhanced comfort.

### SUMMARY OF THE INVENTION

It is an object of the present disclosure to provide a physiological signal monitoring apparatus, which can be detachably mounted on a living body and detect a physiological signal of a user in a manner using a reduced contact area, so as to provide a user with enhanced usage experience.

To achieve at least the above object, the present disclosure provides a physiological signal monitoring apparatus including at least one connection assembly and a physiological signal monitoring device. The connection assembly includes a first connecting body and a second connecting body. The first connecting body and the second connecting body exist in a mutually combined or mutually separated state. The physiological signal monitoring device is detachably combined with a fixing portion through the connection assembly. When the fixing portion is mounted on the living body, the physiological signal monitoring device at least monitors a temperature change and a displacement change of the living body. The physiological signal monitoring device includes at least one engaging member and at least one contact member. A first side of the engaging member at least partially matches with the second connecting body of the connection assembly. The contact member elastically protrudes from an opening of the first side of the engaging member, and is used for temperature sensing. The second connecting body of the connection assembly is used for being fixed at an outer side of a connecting region of the fixing portion, so as to be combined with the first connecting body of the connection assembly. Accordingly, the physiological signal monitoring device can be detachably combined with the fixing portion when the engaging member is detachably connected to the connection assembly. When the fixing portion is mounted on a living body, the contact member is used for being in direct or indirect contact with the living body to perform temperature sensing.

In one embodiment of the present disclosure, the first connecting body of the connection assembly is used for being fixed at an inner side of the connecting region of the fixing portion so as to combine with the second connecting body of the connection assembly. Accordingly, the physiological signal monitoring device becomes combined with the fixing portion when the engaging member is detachably connected to the connection assembly. When the fixing portion is mounted on a living body, the contact member is used for being in direct or indirect contact with the living body to perform temperature sensing.

In one embodiment of the present disclosure, a first side of the second connecting body of the connection assembly at least partially matches with the first side of the engaging member, and has a hole for the contact member to pass through and to come into direct or indirect contact with the living body to perform temperature sensing.

In one embodiment of the present disclosure, the physiological signal monitoring device includes a detection module and a monitoring module. The detection module is used for being detachably combined with the fixing portion through the at least one connection assembly, and outputs detection data when in contact with the living body. The detection module includes: a first temperature detecting unit, including the engaging member and the contact member, for detecting in a direction towards the living body a temperature of the living body and accordingly outputting a first temperature signal; and a second temperature detecting unit, for detecting in a direction apart from the living body a temperature of an ambient environment and accordingly outputting a second temperature signal. The monitoring module, coupled to the detection module, at least receives the first temperature signal and the second temperature signal to monitor the temperature change of the living body and to monitor the displacement change of the living body.

In one embodiment of the present disclosure, the monitoring module includes a displacement sensing unit, a control unit, an output unit, and a wireless transmission unit. The displacement sensing unit detects the displacement change of a body cavity movement of the living body and accordingly generates a displacement signal. The control unit is electrically coupled to the first temperature detecting unit, the second temperature detecting unit, the displacement sensing unit and the output unit. When the control unit detects that the temperature of a living body obtained based on the first temperature signal and the second temperature signal satisfies a temperature alert criterion, the control unit generates a temperature alert signal. When the control unit detects that the displacement change of the living body obtained based on the displacement signal satisfies a displacement alert criterion, the control unit generates a displacement alert signal. The output unit is electrically coupled to the control unit. The wireless transmission unit, electrically coupled to the control unit, is wirelessly connected to a monitoring terminal device, and transmits temperature data based on the first temperature signal and the second temperature signal and displacement data based on the displacement signal to the monitoring terminal device.

In one embodiment of the present disclosure, the first temperature detecting unit further includes a temperature sensor and an elastic member. The temperature sensor is provided in the contact member, and outputs the first temperature signal. The elastic member is engaged with a first end portion of the contact member to cause a second end portion of the contact member to protrude from the opening at the first side of the engaging member.

In one embodiment of the present disclosure, the detection module further comprising a connection housing. The connection housing includes a connecting portion and a plurality of extension portions. The connecting portion at least partially covers an edge of the monitoring module, and is detachably connected to the monitoring module. The extension portions are extended outwards from the connecting portions. The first temperature detecting unit and the second temperature detecting unit are disposed in accommodating spaces in the extension portions, respectively. The extension portions corresponding to the first temperature detecting unit has a first detection opening, which allows the engaging member and the contact member of the first temperature detecting unit to extend from the accommodating space in the extension portion to an exterior of the first detection opening.

In one embodiment of the present disclosure, the extension portion corresponding to the second temperature detecting unit has a second detection opening, which allows a temperature sensor of the second temperature detecting unit to directly or indirectly sense the temperature of the environment from the second detection opening. The first detection opening faces inwards towards a direction for detecting the temperature of the living body, and the second detection opening faces outwards towards a direction for detecting the temperature of the environment.

In one embodiment of the present disclosure, the connecting portion has a plurality of connection openings on an inner side of the connecting portion, and the detection module further includes a plurality of connecting ends. The connecting ends are respectively disposed on at least one of the connecting openings and the inner side of the connecting portion. When the connecting portion is detachably connected to the monitoring module, the monitoring module is electrically coupled through the connecting ends to the first temperature detecting unit and the second temperature detecting unit.

In one embodiment of the present disclosure, the physiological signal monitoring apparatus further includes the fixing portion for mounting on a living body. The fixing portion includes a wearable body, and the connection region is located on the wearable body.

In one embodiment of the present disclosure, the wearable body of the fixing portion includes and is formed by a cleanable material, and can be independently cleaned when the fixing portion and the physiological signal monitoring device are separated.

In one embodiment of the present disclosure, the wearable body of the fixing portion is configured to be secured around and close to a surface of the living body in a manner using a crisscross strap or a hook-and-loop fastener, and to further monitor the temperature change and the displacement change of the living body when the physiological signal monitoring device is detachably combined with the fixing portion.

Accordingly, with the above embodiments of the physiological signal monitoring apparatus, the physiological signal monitoring apparatus can be detachably mounted on a living body and detect a physiological signal of a user in a manner using a reduced contact area, providing a user with enhanced usage experience.

### BRIEF DESCRIPTION OF THE DRAWINGS

FIG. 1 is a schematic diagram of a physiological signal monitoring apparatus according to an embodiment of the present disclosure;

FIG. 2 is a schematic diagram of a physiological signal monitoring apparatus to be combined according to an embodiment of the present disclosure;

FIG. 3 is a schematic diagram of a connection assembly at a fixing portion according to an embodiment of the present disclosure;

FIG. 4 is a schematic diagram of a connection assembly according to an embodiment of the present disclosure;

FIG. 5 is a schematic diagram of a physiological signal monitoring device detachably combined with a fixing portion through a connection assembly according to an embodiment;

FIG. 6 is a schematic diagram of a physiological signal monitoring device detachably combined with a fixing portion through a connection assembly according to an embodiment;

FIG. 7 is a schematic diagram of a connection assembly combined with a fixing portion according to an embodiment of the present disclosure;

FIG. 8 is a schematic diagram of a connection assembly according to another embodiment;

FIG. 9 is a schematic diagram of a physiological signal monitoring device detachably combined with a fixing portion through a connection assembly according to another embodiment;

FIG. 10 is a block diagram of a physiological signal monitoring device according to an embodiment;

FIG. 11 is a block diagram of a physiological signal monitoring device communicating with a monitoring terminal device;

FIG. 12 is a schematic diagram of an engaging member according to an embodiment;

FIG. 13 is a section view of the engaging member in FIG. 12;

FIG. 14 is an exploded schematic diagram of a first temperature detecting unit according to an embodiment;

FIG. 15 is a section schematic diagram of the first temperature detecting unit according to an embodiment;

FIG. 16 is a top side view of a physiological signal monitoring device according to an embodiment;

FIG. 17 is a bottom view of the physiological signal monitoring device in FIG. 16;

FIG. 18 is a front view of the physiological signal monitoring device in FIG. 16;

FIG. 19 is a rear view of the physiological signal monitoring device in FIG. 16;

FIG. 20 is a schematic diagram of the physiological signal monitoring device in FIG. 16 combined with a connection assembly and a fixing portion;

FIG. 21 is a schematic diagram of a detection module of the physiological signal monitoring device in FIG. 16 according to an embodiment;

FIG. 22 is a schematic diagram of a monitoring module of the physiological signal monitoring device in FIG. 16 according to an embodiment; and

FIG. 23 is a schematic diagram of the detection module in FIG. 21 being disassembled.

### DETAILED DESCRIPTION OF THE PREFERRED EMBODIMENTS

To thoroughly understand the objects, characteristics and effects of the present disclosure, the present disclosure is described in detail by the following embodiments in conjunction with the accompanying drawings below.

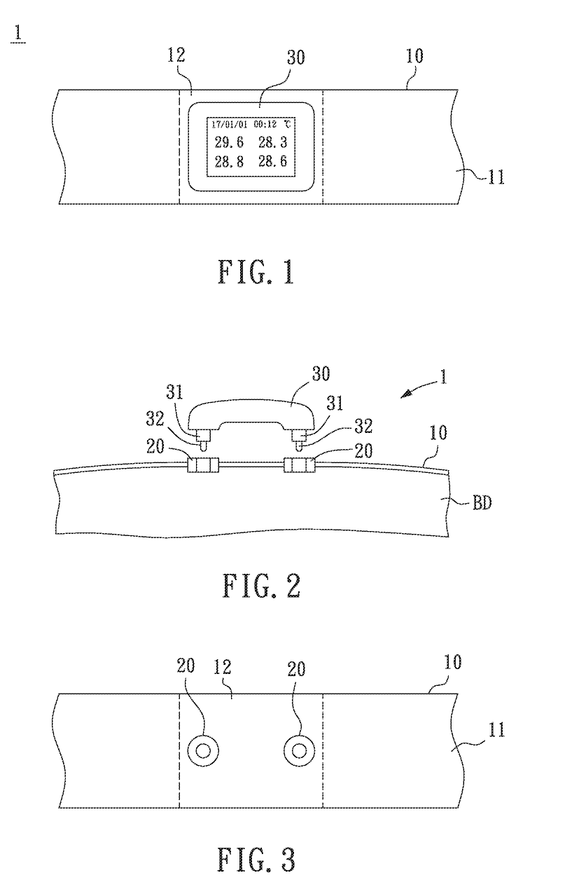

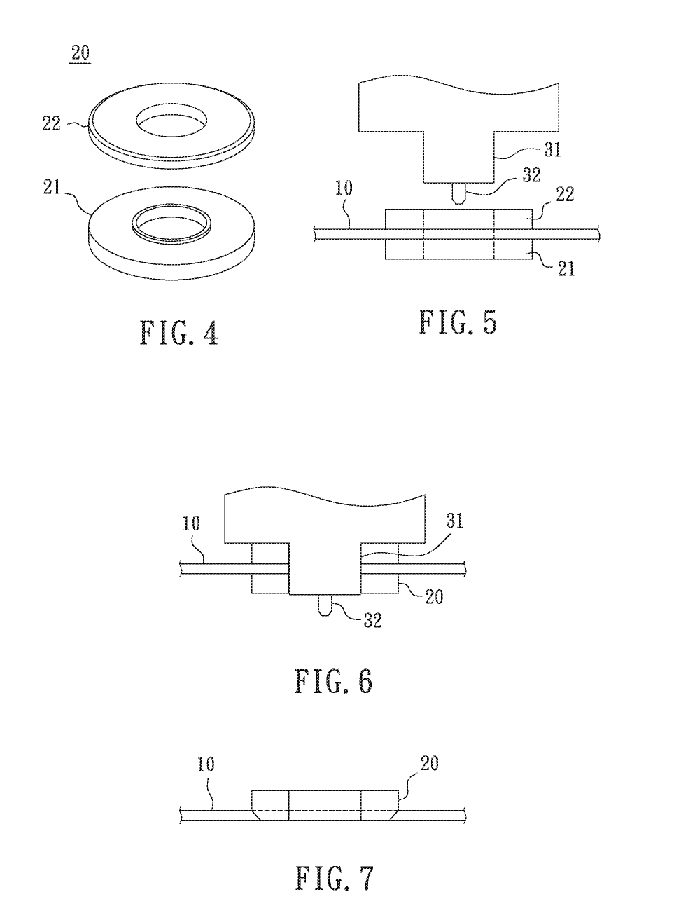

Refer to FIGS. 1 to 4. FIG. 1 is a schematic diagram of a physiological signal monitoring apparatus according to an embodiment of the present disclosure. FIG. 2 is a schematic diagram of a physiological signal monitoring apparatus to be combined according to an embodiment of the present disclosure. FIG. 3 is a schematic diagram of a connection assembly 20 at a fixing portion 10 according to an embodiment of the present disclosure. FIG. 4 is a schematic diagram of the connection assembly 20 according to an embodiment of the present disclosure. As shown in FIGS. 1 to 3, the physiological signal monitoring apparatus 1 includes at least one connection assembly 20 and a physiological signal monitoring device 30.

As shown in FIGS. 3 and 4, the connection assembly 20 includes a first connecting body 21 and a second connecting body 22. The first connecting body 21 and the second connecting body 22 are in a mutually combined or mutually separated state. For example, the first connecting body 21 and the second connecting body 22 have respective recessed/protruding (e.g., notched) or mutually engaging structures so as to be mutually combined. Further, each of the first connecting body 21 and the second connecting body 22 has at least one hole.

As shown in FIGS. 1 and 2, the physiological signal monitoring device 30 can be detachably combined with a fixing portion 10 through the connection assembly 20. When the fixing portion 10 is mounted on a living body BD, the physiological signal monitoring device 30 at least monitors a temperature change and a displacement change of the living body BD. The fixing portion 10 includes a wearable body 11 and a connection region 12 for mounting on the living body BD. For example, the fixing portion 10 is an object that can be mounted on the living body BD, such as a wearable object. For example, the living body BD is a human body, e.g., a baby, a teenager, an adult or an elder person, or an animal, a mammal, e.g., a pet such as a cat or a dog, or any kind of livestock such as a horse or a cow, or other animals. It should be noted that the implementation of the present disclosure is not limited to the above examples of the fixing portion and the detection target.

As shown in FIGS. 2, 5 and 6, the physiological signal monitoring device 30 includes at least one engaging member 31 and at least one contact member 32. A first side of the engaging member 31 at least partially matches with the second connecting body 22 of the connection assembly 20. The contact member 32 elastically protrudes from an opening at the first side of the engaging member 31 and is used for temperature sensing.

As shown in FIGS. 2, 5 and 6, the second connecting body 22 of the connection assembly 20 is fixed at an outer side of the connection region 12 of the fixing portion 10 so as to combine with the first connecting body 21 of the connection assembly 20. Thus, the physiological signal monitoring device 30 can be detachably combined with the fixing portion 10 by detachably connecting the engaging member 31 to the connection assembly 20. Further, when the fixing portion 10 is mounted on the living body, the contact member 32 comes into direct or indirect contact with the living body to perform temperature sensing.

As shown in FIGS. 2, 5 and 6, the first connecting body 21 of the connection assembly 20 is fixed at an inner side of the connection region 12 of the fixing portion 10 so as to combine with the second connecting body 22 of the connection assembly 20. Thus, the physiological signal monitoring device 30 can be combined with the fixing portion 10 by detachably connecting the engaging member 31 to the connection assembly 20. Further, when the fixing portion 10 is mounted on the living body, the contact member 32 comes into direct or indirect contact with the living body to perform temperature sensing. Further, as shown in FIGS. 3 and 5, a region of the fixing portion 10 corresponding to the hole of the connection assembly 20 is also provided with a hole for the engaging member 31 to pass through.

FIG. 7 shows a schematic diagram of the connection assembly 20 to be combined with the fixing portion 10 according to an embodiment. Compared to FIG. 5, the connection assembly 20 in FIG. 7 may also be fixed at the outer side of the fixing portion 10 of the connection assembly 20. In FIG. 7, the fixing portion 10 may be any wearable fabric, and a user may fix the connection assembly 20 at the outer side of the fixing portion 10 by way of sewing. For another example, the fixing portion 10 in FIG. 7 may be any wearable object, and the connection assembly 20 is fixed at the fixing portion 10 by way of implanting or embedding.

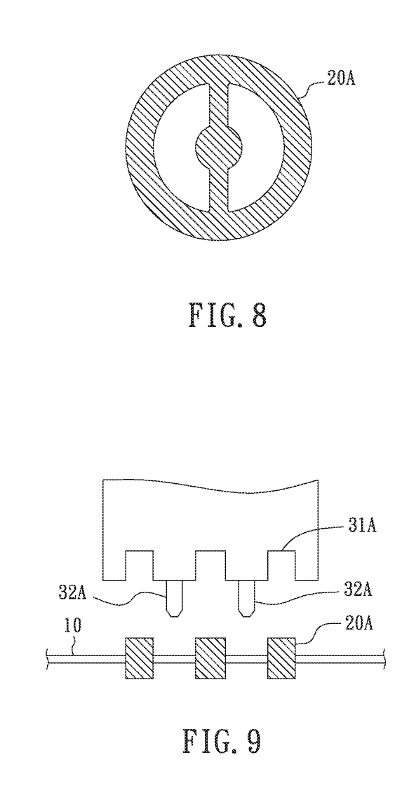

For example, a first side of the second connecting body 22 of the connection assembly 20 at least partially matches with the first side of the engaging member 31, and has at least one hole for the contact member 32 to pass through to come into direct or indirect contact with the living body to perform temperature sensing. Further, FIG. 8 shows a schematic diagram of a connection assembly according to another embodiment. Referring to FIG. 8, a connection assembly 20A has two holes for the contact member to pass through. FIG. 9 shows a schematic diagram of a physiological signal monitoring device detachable combined with the fixing portion 10 through the assembly connection 20A according to another embodiment. As shown in FIG. 9, the physiological signal monitoring device includes an engaging member 31A, at least in part, in a recessed and protruding (e.g., notched) shape matching with the connection assembly 20A and two contact members 32A.

In the implementation of any of the above embodiments, the connection assembly may also be implemented in other manners. For example, the connection assembly may include a first connecting body and a second connecting body. The second connecting body has a hole for engaging with the engaging member, and the first connecting body does not have a through hole corresponding to the hole of the second connecting body. Thus, when the first connecting body and the second connecting body are combined at the fixing portion 10, one side of the first connecting body may come into contact with the living body BD. By this example of the connection assembly, given that the engaging member 31 and the contact member 32 of the physiological signal monitoring device 30 are appropriately configured (e.g., by changing the lengths of the two), the engaging member 31 is provided with a securing effect through the second connecting body of the connection assembly, and the contact member 32 can further come into contact with the first connecting body of the connection assembly. Accordingly, the side of the first connecting body that is in contact with the living body BD can be used as a sensing region extended from the contact member 32, wherein the first connecting body is a component of a metal or an electrically conductive or heat conductive material. Furthermore, in other examples based on FIG. 6 or 9, at the outer side of the first connecting body on the lower portion of the Figure, the connection assembly may further include an extension body capable of being in contact with the contact member 32. The extension body can sleeve around the first connecting body 20 to serve the function of a sensing region extended from the contact member 32.

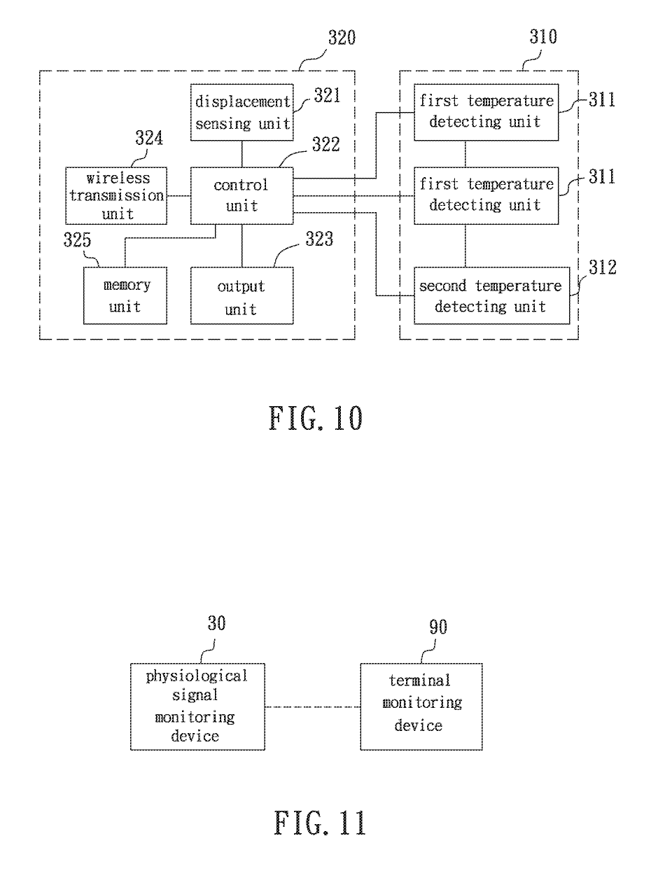

FIG. 10 shows a block diagram of the physiological signal monitoring device 30 according to an embodiment of the present disclosure. As shown in FIG. 10, the physiological signal monitoring device 30 includes a detection module 310 and a monitoring module 320.

The detection module 310 can be detachably combined with the fixing portion 10 through the at least one connection assembly 20, and can output detection data when in contact with a living body. The detection module 310 includes at least one first temperature detecting unit 311 and at least one second temperature detecting unit 312. The first temperature detecting unit 311 includes the engaging member 31 and the contact member 32, and detects a temperature of the living body in a direction towards the living body and accordingly outputs a first temperature signal. The second temperature detecting unit 312 detects a temperature of an ambient environment in a direction apart from the living body and accordingly outputs a second temperature signal.

The monitoring module 320, coupled to the detection module 310, at least receives the first temperature signal and the second temperature signal so as to monitor a temperature change of the living body and to monitor a displacement change of the living body. As shown in FIG. 10, the monitoring module 320 includes a displacement sensing unit 321, a control unit 322, an output unit 323 and a wireless transmission unit 324. The monitoring module 320 can further communicate with an external device in a wireless manner, so as to transmit the monitored temperature change and displacement change to the external device, as shown in FIG. 11.

The displacement sensing unit 321 detects a displacement change of a body cavity movement of the living body and accordingly generates a displacement signal. For example, the displacement sensing unit 321 may include an accelerometer or gyroscope.

The control unit 322 is electrically coupled to the first temperature detecting unit 311, the second temperature detecting unit 312, the displacement sensing unit 321 and the output unit 323. When the control unit 322 detects that the temperature of the living body obtained based on the first temperature signal and the second temperature signal satisfies a temperature alert criterion, the control unit 322 generates a temperature alert signal. When the control unit 322 detects that the displacement change of the living body obtained based on the displacement signal satisfies a displacement alert criterion, the control unit 322 generates a displacement alert signal. For example, the temperature alert criterion is that when the temperature of the living body is greater than an upper temperature threshold, e.g., greater than 38° C., a temperature alert signal is generated, or is that when the temperature of the living body is less than a lower temperature threshold, e.g., less than 37° C., a temperature alert signal is generated. For example, the displacement alert criterion is that when the displacement of the living body is greater than an upper displacement threshold, a displacement alert signal is generated, or is that when the displacement of the living body is less than a lower displacement threshold, a displacement alert signal is generated. For another example, the detection module 310 may include a plurality of first temperature detecting units 311 so as to accordingly obtain a plurality of temperature values representing the living body. Thus, the control unit 322 can obtain an estimated value, a maximum value or a minimum value of the temperature of the living body by using statistical calculation or an average value within a unit of time, so as to determine whether the temperature of the living body is abnormal. Further, for example, the displacement sensing unit 321 may output variations in displacement in three or more axial measurements, and the control unit 322 can represent the displacement value of the living body based on the displacement change in three coordinate axes (e.g., a sum of absolute values or a sum of squares of the displacement changes of the three coordinate axes). However, the present disclosure is not limited to the above examples.

The output unit 323, electrically coupled to the control unit 322, is a display device such as an LCD, an electronic paper or OLED, and is capable of displaying data such as the detected temperature, environment temperature, displacement, or an alert signal. Alternatively, a user interface may also be used to allow a user to easily operate or configure the physiological signal monitoring device 30.

The wireless transmission unit 324, electrically coupled to the control unit 322, is wirelessly linked to a monitoring terminal device 90 and transmits the temperature data based on the first temperature signal and the second temperature signal and the displacement data based on the displacement signal to the monitoring terminal device 90, as shown in FIG. 11. For example, the wireless transmission unit 324 supports Bluetooth, Bluetooth Low Energy (BLE), infrared, Zigbee or other wireless communication protocols.

Further, the monitoring module 320 may include other components based on requirements, e.g., a memory unit 325 for storing input or output data from other units or an external device, or may be configured to be operable by the monitoring module 320, or, e.g., a wired communication unit such as a USB connection circuit, a power circuit, a rechargeable battery, a solar battery, an alert light or a beeper. Further, for example, one of the monitoring module 320 and the detection module 310 may be provided with other sensors, e.g., a heart rate sensor. However, the present disclosure is not limited to the above examples.

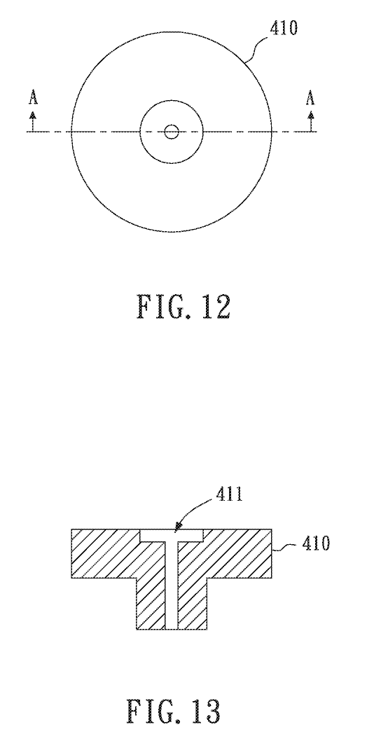

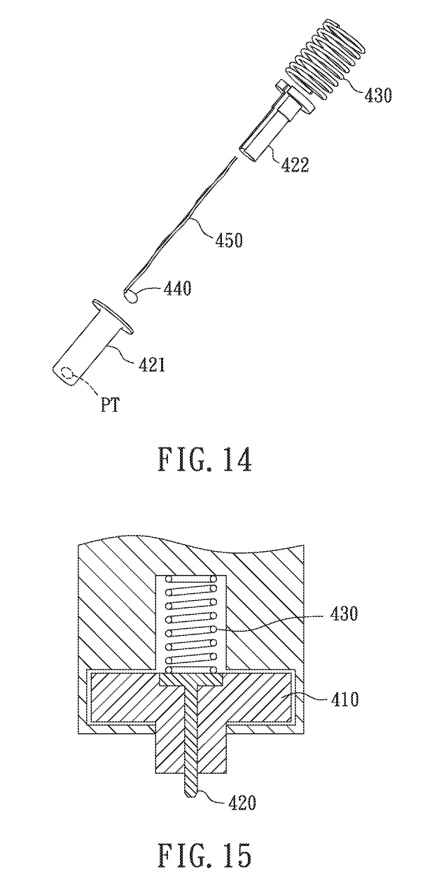

Various implementations of an internal structure of the first temperature detecting unit 311 of the detection module 310 are given with the examples below. As previously described, the first temperature detecting unit 311 includes the engaging member 31 and the contact member 32. FIG. 12 shows a schematic diagram of an engaging member according to an embodiment. FIG. 13 shows a section view of an engaging member 410 along a line A-A in FIG. 12. As shown in FIG. 13, a lower portion of the engaging member 410 has a protrusion, and the engaging member 31 has a hole 411 for accommodating a contact member 420. FIG. 14 shows an exploded schematic diagram of the first temperature detecting unit 311 according to an embodiment. FIG. 15 shows a section schematic diagram of the first temperature detecting unit 311 according to an embodiment. As shown in FIG. 14, the first temperature detecting unit 311 further includes an elastic member 430 and a temperature sensor 440. The temperature sensor 440 is disposed in the contact member 420, and outputs the first temperature signal. For example, in FIG. 14, the contact member 32 includes a first sub contact member 421 and a second sub contact member 422. When the temperature sensor 440 is placed between the first sub contact member 421 and the second sub contact member 422, and the first sub contact member 421 and the second sub contact member 422 are combined, the temperature sensor 440 is placed in the first sub contact member 421 as shown in FIG. 14, at a position PT indicated by a dotted ellipse. Two signal lines 450 of the temperature sensor 440 can pass through a hole of the second sub contact member 422 so as to be guided to two contact ends and to be electrically coupled to the monitoring module 320. As shown in FIGS. 14 and 15, the elastic member 430 is engaged with a first end portion of the contact member 420, such that a second end portion of the contact member 420 can protrude from an opening at a first side of the engaging member 410.

Other implementations of the physiological signal monitoring device 30 will be further exemplified below.

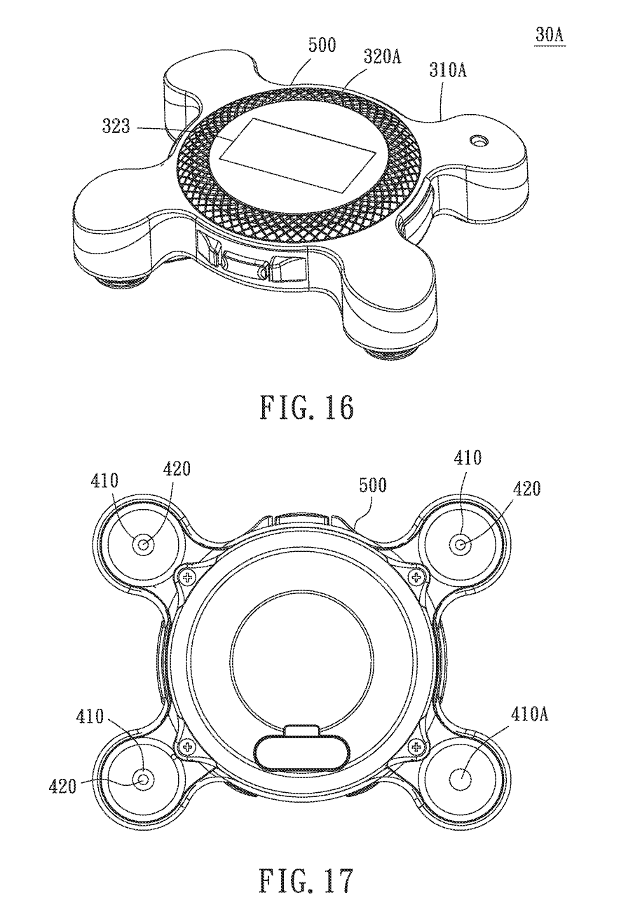

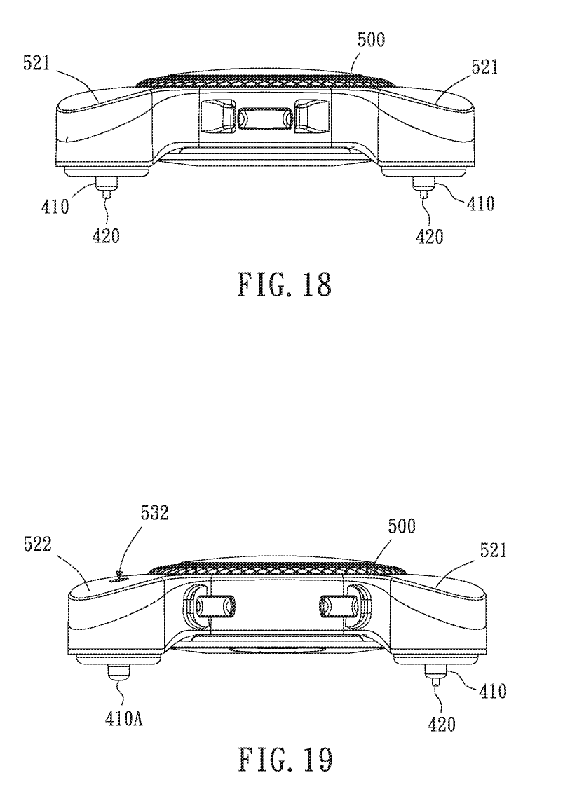

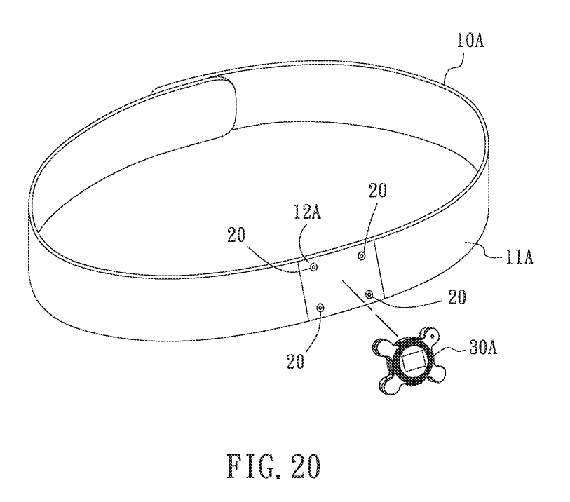

Referring to FIGS. 16 to 20 showing a physiological signal monitoring device 30 according to another embodiment. FIG. 16 is a top side view of the physiological signal monitoring device according to another embodiment. FIG. 17 is a bottom view of a physiological signal monitoring device 30A in FIG. 16. FIG. 18 is a front view of the physiological signal monitoring device 30A in FIG. 16. FIG. 19 is a rear view of the physiological signal monitoring device 30A in FIG. 16. FIG. 20 is a schematic diagram of the physiological signal 30A in FIG. 16 combined with the connection assembly 20 and a fixing portion 10A.

In some embodiments of the physiological signal monitoring apparatus of the present disclosure, the physiological signal monitoring apparatus may include a fixing portion (e.g., 10 or 10A), and a wearable portion (e.g., 11 or 11A) of the fixing portion includes and is formed by (at least in part or in whole) a cleanable material. For example, when the fixing portion is separated from the physiological signal monitoring device (e.g., 30 or 30A), the fixing portion can be independently cleaned. Further, for example, the wearable body of the fixing portion is configured to be secured around and close to a surface of the living body, e.g., a chest, an abdomen, a hand or other parts, in a manner using a crisscross strap or a hook-and-loop fastener. Thus, the physiological signal monitoring device can be detachably combined with the fixing portion to monitor the temperature change and the displacement change of the living body. However, the present disclosure is not limited to the examples of the fixing portion. That is, when the physiological signal monitoring apparatus is implemented or sold, the fixing portion may be regarded as an environmental part or an option according to a user's requirement or a specification requirement of a product to be sold.

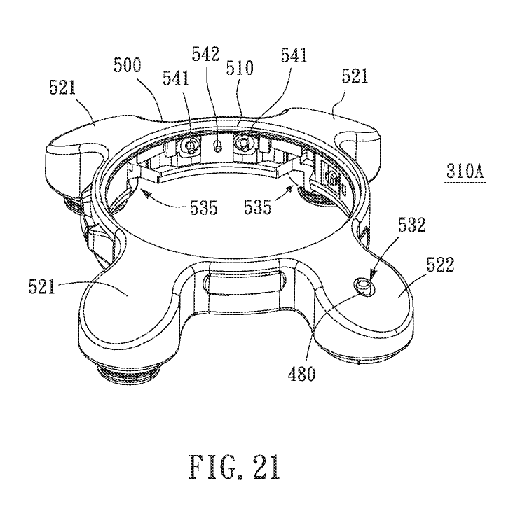

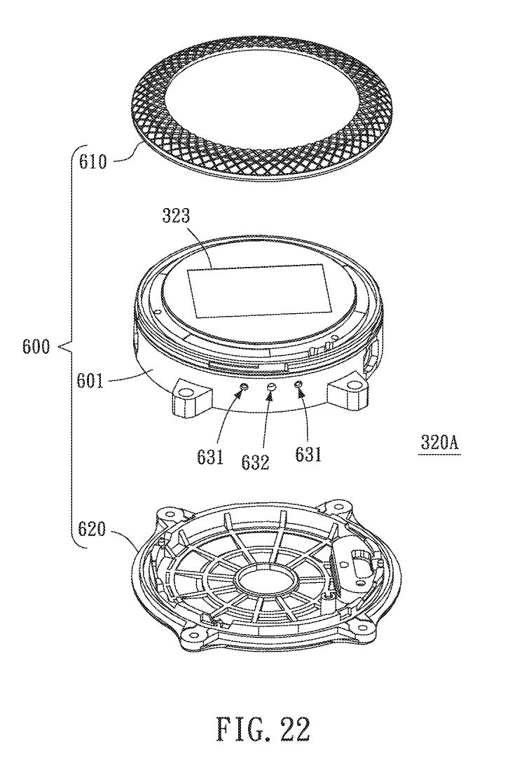

Further, in the embodiment of the physiological signal monitoring device 30A in FIG. 16, a detection module 310A and a monitoring module 320A of the physiological signal monitoring device 30A can be configured as detachably combined structures and having a detachable electrical coupling relationship. Thus, maintenance and repairs can be readily performed, or updating or upgrading of hardware and software can be facilitated. FIGS. 21 and 22 show schematic diagrams of the detection module 310A and the monitoring module 320A of the physiological signal monitoring device 30A in FIG. 16 implemented as being detachable according to an embodiment. As shown in FIG. 21, the detection module 310A further includes a connection housing 500. The connection housing 500 includes a connecting portion 510 and a plurality of extension portions 521 and 522. As shown in FIG. 22, a housing 600 of the monitoring module 320A includes an upper cover 610, a side cover 601 and a lower cover 620.

As shown in FIG. 21, the connecting portion 510 at least partially covers or completely covers an edge of the monitoring module 320A, and can be detachably connected to or engaged with the monitoring module 320A. For example, the connecting portion 510 has a ring shape, and covers the side cover 601 at an edge of the housing 600 of the monitoring module 320A shown in FIG. 22. The extension portions 521 extend outwards from the connecting portion 510, and a plurality of first temperature detecting units 311 of the detection module 310A are respectively disposed in the accommodating spaces in the extension portions 521. A second temperature detecting unit 312 of the detection module 310A is disposed in an accommodating space of the extension portion 522.

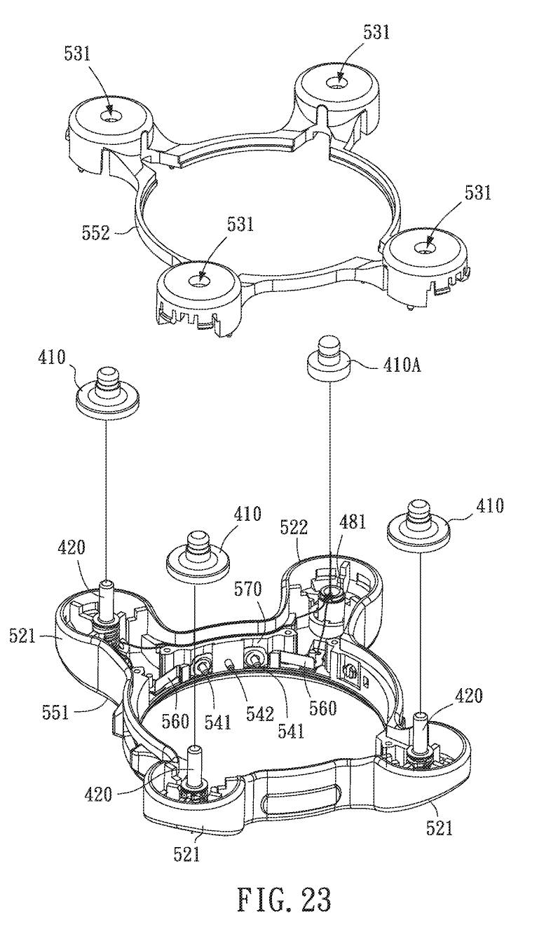

FIG. 23 shows a schematic diagram of the detection module 310A in FIG. 21 being disassembled according to an embodiment. Again referring to FIGS. 17, 21 and 23, in one embodiment, the extension portion 521 corresponding to the first temperature detecting unit 311 has a first detection opening 531, which allows the engaging member 410 and contact member 420 of the first temperature detecting unit 311 to extend from the accommodating space in the extension portion 521 to out of the first detection opening 531.

Referring to FIGS. 19, 21 and 23, in one embodiment, the extension portion 522 corresponding to the second temperature detecting unit 312 has a second detection opening 532, which allows a temperature sensor 481 of the second temperature detecting unit 312 to sense the temperature of the environment indirectly through a sensing element 480 or directly from the second detection opening 532. The first detection opening 531 faces a direction towards an interior for detecting the temperature of the living body, and the second detection opening 532 faces a direction towards an exterior for detecting the temperature of the environment. Further, as shown in FIGS. 19 and 23, a lower part of the extension portion 522 may also be provided with a first detection opening 531 to allow an engaging member 410A protruding from the first detection opening 531 so as to be combined with the connection assembly 20, wherein the engaging member 410A does not have any holes. However, the above is an optional implementation, and the present disclosure is not limited to such example.

As shown in FIGS. 21 and 23, a plurality of connecting openings 535 are provided at an inner side of the connecting portion 510, and the detection module 310A further includes a plurality of connecting ends (e.g., 541 and 542). The connecting ends are respectively disposed on at least one of the connecting openings 535 and an inner side of the connecting portion 510. For example, a part or all of the connecting ends may be disposed in the connection openings 535 or at the inner side of the connecting portion 510. When the connecting portion 510 is detachably connected to the monitoring module 320A, the monitoring module 320A is electrically coupled with the first temperature detecting unit 311 and the second temperature detecting unit 312 through the connecting ends.

Further, as shown in FIG. 23, the connection housing 500 may be implemented to include an upper housing portion 551 and a lower housing portion 552. Referring to FIGS. 21 and 23, the connecting ends 541 and 542 of the detection module 310A are disposed at an inner side of the upper housing portion 551 (which may be regarded as the inner side of the connecting portion 510). For example, the connecting end 541 is used for temperature signal transmission, and the connecting end 542 is used for grounding. When the detection module 310A and the monitoring module 320A are detachably engaged, the connecting ends 541 and 542 of the detection module 310A may be electrically coupled to connecting ends 631 and 632 of the monitoring module 320A in FIG. 22, respectively. The connecting ends 631 and 632 may be configured on the hole of the housing or may protrude out of the hole. As shown in FIG. 23, an output of the first temperature detecting unit 311 (or the second temperature detecting unit 312) is electrically coupled to the corresponding connecting end 541 through a conductive member 560 disposed near the connection opening 535. For example, the first temperature detecting unit 311 and the second temperature detecting unit 312 may be further configured to have a common ground node, which is electrically coupled to the connecting end 542. For example, as shown in FIG. 23, a separating member 570 is disposed at an inner side of the upper housing portion 551, so that the conductive member 560, the connecting ends 541 and 542, and the connecting lines for grounding are free from mutual interference. Thus, the accommodating space provided by the connection housing 500 can be effectively utilized, and the physiological signal monitoring device 30A can then achieve the mechanical coupling and electrical coupling structures of the detection module 310A and the monitoring module 320A in a reduced volume. However, the present disclosure is not limited to the implementations of the connecting ends of the above example, and the connecting ends may be implemented by other methods. For example, the connecting ends may be disposed at the connection opening 535, thus similarly achieving the above mechanical coupling and electrical coupling effects.

As described in the various embodiments of the physiological signal monitoring apparatus, the physiological signal monitoring apparatus can be detachably mounted on a living body, and can detect physiological signals of a user in a manner using a reduced contact area by using the contact member that is employed to come into contact with the living body, providing a user with enhance usage experience. For example, when the physiological signal monitoring apparatus is applied for fulfilling detection requirements for babies, children or elder persons, the physiological signal monitoring device provides enhanced comfort as well as enhanced usage experience to a user in a scenario where physiological signals need to be detected for over an extended period of time.

While the disclosure has been described by way of example and in terms of the preferred embodiments, it is to be understood that the disclosure is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. A physiological signal monitoring apparatus, comprising:
at least one connection assembly, comprising a first connecting body and a second connecting body, the first connecting body and the second connecting body existing in a mutually combined or mutually separated state;
a physiological signal monitoring device, detachably combined with a fixing portion through the connection assembly, at least for monitoring a temperature change and a displacement change of a living body when the fixing portion is mounted on the living body; the physiological signal monitoring device comprising at least one engaging member and at least one contact member; a first side of the engaging member at least partially matching with the second connecting body of the connection assembly, the contact member elastically protruding from an opening at the first side of the engaging member and used for temperature sensing;
wherein the second connecting body of the connection assembly is used for being fixed at an outer side of a connecting region of the fixing portion so as to be combined with the first connecting body of the connection assembly, such that the physiological signal monitoring device is detachably combined with the fixing portion when the engaging member is detachably connected to the connection assembly; when the fixing portion is mounted on the living body, the contact member is used for being in direct or indirect contact with the living body to perform temperature sensing.

2. The physiological signal monitoring apparatus according to claim 1, wherein the first connecting body of the connection assembly is used for being fixed at an inner side of a connection region of the fixing portion so as to be combined with the second connecting body of the connection assembly, such that the physiological signal monitoring device becomes combined with the fixing portion when the engaging member is detachably connected to the connection assembly; when the fixing portion is mounted on the living body, the contact member is used for being in direct or indirect contact with the living body to perform temperature sensing.

3. The physiological signal monitoring apparatus according to claim 2, wherein a first side of the second connecting body of the connection assembly at least partially matches with the first side of the engaging member, and has a hole for the contact member to pass through and to come into direct or indirect contact with the living body to perform temperature sensing.

4. The physiological signal monitoring apparatus according to claim 1, wherein a first side of the second connecting body of the connection assembly at least partially matches with the first side of the engaging member, and has a hole for the contact member to pass through and to come into direct or indirect contact with the living body to perform temperature sensing.

5. The physiological signal monitoring apparatus according to claim 1, wherein the physiological signal monitoring device comprises:
a detection module, for being detachably combined with the fixing portion through the at least one connection assembly, and outputting detection data when in contact with the living body, the detection module comprising:
a first temperature sensing unit, comprising the engaging member and the contact member, for detecting a temperature of the living body in a direction towards the living body and accordingly outputting a first temperature signal; and
a second temperature detecting unit, for detecting a temperature of an ambient temperature in a direction apart from the living body and accordingly outputting a second temperature signal; and
a monitoring module, coupled to the detection module, for receiving the first temperature signal and the second temperature signal to monitor a temperature change of the living body and a displacement change of the living body.

6. The physiological signal monitoring apparatus according to claim 5, wherein the monitoring module comprises:
a displacement sensing unit, for detecting the displacement change of a body cavity movement of the living body and accordingly generating a displacement signal;
a control unit, electrically coupled to the first temperature sensing unit, the second temperature sensing unit, the displacement sensing unit and the output unit; wherein the control unit generates a temperature alert signal when the control unit detects that the temperature of the living body obtained based on the first temperature signal and the second temperature signal satisfies a temperature alert criterion, and generates a displacement alert signal when the control unit detects that the displacement change of the living body obtained based on the displacement signal satisfies a displacement alert criterion;
an output unit, electrically coupled to the control unit; and
a wireless transmission unit, electrically coupled to the control unit, for wirelessly connecting to a monitoring terminal device, and transmitting temperature data based on the first temperature signal and the second temperature signal and displacement data based on the displacement signal to the monitoring terminal device.

7. The physiological signal monitoring apparatus according to claim 5, wherein the detection module further comprises:
a connection housing, comprising:
a connecting portion, for at least partially covering an edge of the monitoring module, and detachably connected to the monitoring module; and
a plurality of extension portions, extending outwards from the connecting portion, wherein the first temperature detecting unit and the second temperature detecting unit are respectively disposed in accommodation spaces in the extension portions;
wherein the extension portion corresponding to the first temperature sensing unit has a first detection opening for the engaging member and the contact member of the first temperature sensing unit to extend from the accommodating space of the extension portion to out of the first detection opening.

8. The physiological signal monitoring apparatus according to claim 7, wherein the extension portion corresponding to the second temperature sensing unit has a second detection opening for a temperature sensor of the second temperature detecting unit to directly or indirectly sense a temperature of an environment; the first detection opening faces inwards towards a direction for detecting the temperature of the living body, and the second detection opening faces outwards towards a direction for detecting the temperature of the environment.

9. The physiological signal monitoring apparatus according to claim 7, wherein the connecting portion has a plurality of connection openings at an inner side of the connecting portion, the detection module further comprises a plurality of connecting ends, and the connecting ends are respectively disposed on at least one of the connection openings and the inner side of the connecting portion; when the connecting portion is detachably connected to the monitoring module, the monitoring module is electrically coupled to the first temperature detecting unit and the second temperature detecting unit through the connecting ends.

10. The physiological signal monitoring apparatus according to claim 5, wherein the first temperature detecting unit further comprises:
a temperature sensor, disposed in the contact member, for outputting the first temperature signal; and
an elastic member, engaged with a first end portion of the contact member to allow a second end portion of the contact member to protrude from an opening at the first side of the engaging member.

11. The physiological signal monitoring apparatus according to claim 10, wherein the detection module further comprises:
a connection housing, comprising:
a connecting portion, for at least partially covering an edge of the monitoring module, and detachably connected to the monitoring module; and
a plurality of extension portions, extending outwards from the connecting portion, wherein the first temperature detecting unit and the second temperature detecting unit are respectively disposed in accommodating spaces in the extension portions;
wherein the extension portion corresponding to the first temperature sensing unit has a first detection opening for the engaging member and the contact member of the first temperature sensing unit to extend from the accommodating space of the extension portion to out of the first detection opening.

12. The physiological signal monitoring apparatus according to claim 11, wherein the extension portion corresponding to the second temperature sensing unit has a second detection opening for a temperature sensor of the second temperature detecting unit to directly or indirectly sense a temperature of an environment; the first detection opening faces inwards towards a direction for detecting the temperature of the living body, and the second detection opening faces outwards towards a direction for detecting the temperature of the environment.

13. The physiological signal monitoring apparatus according to claim 11, wherein the connecting portion has a plurality of connection openings on an inner side of the connecting portion, the detection module further comprises a plurality of connecting ends, and the connecting ends are respectively disposed on at least one of the connection openings and the inner side of the connecting portion; when the connecting portion is detachably connected to the monitoring module, the monitoring module is electrically coupled to the first temperature detecting unit and the second temperature detecting unit through the connecting ends.

14. The physiological signal monitoring apparatus according to claim 1, further comprising the fixing portion; wherein the fixing portion is for mounting on a living body, and comprises a wearable body; the connection region is located on the wearable body.

15. The physiological signal monitoring apparatus according to claim 14, wherein the wearable body of the fixing portion comprises a cleanable material, and is capable of being independently cleaned when the fixing portion and the physiological signal monitoring device are separated.

16. The physiological signal monitoring apparatus according to claim 14, wherein the wearable body of the fixing portion is configured to be secured around and close to a surface of the living body in a manner using a crisscross strap or a hook-and-loop fastener, so that the physiological signal monitoring device, when detachably combined with the fixing portion, monitors the temperature change and the displacement change of the living body.
